SEQUENCE LISTING (13 sequences)

SEQ ID NO: 1 - protein, 1070 aa, Plasmodium vivax
MKGKNRSLFVLLVLLLLHKVNNVLLERTIETLLECKNEYVKGENGYKLAKGHHCVEEDNLERWLQGTNERRSEENIKYKYGVTELKIKYAQMNGKRSSRILKESIYGAHNFGGNSYMEGKDGGDKTGEEKDGEHKTDSKTDNGKGANNLVMLDYETSSNGQPAGTLDNVLEFVTGHEGNSRKNSSNGGNPYDIDHKKTISSAIINHAFLQNTVMKNCNYKRKRRERDWDCNTKKDVCIPDRRYQLCMKELTNLVNNTDTNFHRDITFRKLYLKRKLIYDAAVEGDLLLKLNNYRYNKDFCKDIRWSLGDFGDIIMGTDMEGIGYSKVVENNLRSIFGTDEKAQQRRKQWWNESKAQIWTAMMYSVKKRLKGNFIWICKLNVAVNIEPQIYRWIREWGRDYVSELPTEVQKLKEKCDGKINYTDKKVCKVPPCQNACKSYDQWITRKKNQWDVLSNKFISVKNAEKVQTAGIVTPYDILKQELDEFNEVAFENEINKRDGAYIELCVCSVEEAKKNTQEVVTNVDNAAKSQATNSNPISQPVDSSKAEKVPGDSTHGNVNSGQDSSTTGKAVTGDGQNGNQTPAESDVQRSDIAESVSAKNVDPQKSVSKRSDDTASVTGIAEAGKENLGASNSRPSESTVEANSPGDDTVNSASIPVVSGENPLVTPYNGLRHSKDNSDSDGPAESMANPDSNSKGETGKGQDNDMAKATKDSSNSSDGTSSATGDTTDAVDREINKGVPEDRDKTVGSKDGGGEDNSANKDAATVVGEDRIRENSAGGSTNDRSKNDTEKNGASTPDSKQSEDATALSKTESLESTESGDRTTNDTTNSLENKNGGKEKDLQKHDFKSNDTPNEEPNSDQTTDAEGHDRDSIKNDKAERRKHMNKDTFTKNTNSHHLNSNNNLSNGKLDIKEYKYRDVKATREDIILMSSVRKCNNNISLEYCNSVEDKISSNTCSREKSKNLCCSISDFCLNYFDVYSYEYLSCMKKEFEDPSYKCFTKGGFKDKTYFAAAGALLILLLLIASRKMIKNDSEEATFNEFEEYCDNIHRIPLMPNNIEHMQPSTPLDYS

SEQ ID NO: 2 - protein, 308 aa, Plasmodium vivax
DHKKTISSAIINHAFLQNTVMKNCNYKRKRRERDWDCNTKKDVCIPDRRYQLCMKELTNLVNNTDTNFHRDITFRKLYLKRKLIYDAAVEGDLLLKLNNYRYNKDFCKDIRWSLGDFGDIIMGTDMEGIGYSKVVENNLRSIFGTDEKAQQRRKQWWNESKAQIWTAMMYSVKKRLKGNFIWICKLNVAVNIEPQIYRWIREWGRDYVSELPTEVQKLKEKCDGKINYTDKKVCKVPPCQNACKSYDQWITRKKNQWDVLSNKFISVKNAEKVQTAGIVTPYDILKQELDEFNEVAFENEINKRDGAY

SEQ ID NO: 3 - protein, 510 aa, Plasmodium vivax
ERDWDCNTKKDVCIPDRRYQLCMKELTNLVNNTDTNFHRDITFRKLYLKRKLIYDAAVEGDLLLKLNNYRYNKDFCKDIRWSLGDFGDIIMGTDMEGIGYSKVVENNLRSIFGTGEKAQQHRKQWWNESKAQIWTAMMYSVKKRLKGNFIWICKINVAVNIEPQIYRWIREWGRDYVSELPTEVQKLKEKCDGKINYTDKKVCKVPPCQNACKSYDQWITRKKNQWDVLSNKFKSVKNAEKVQTAGIVTPYDILKQELDEFNEVAFENEINKRDGAYIELCVCSVEEAKKNTQEVVTNVDNAAKSQATNSNPISQPVDSSKAEKVPGDSTHGNVNSGQDSSTTGKAVTGDGQNGNQTPAESDVQRSDIAESVSAKNVDPQKSVSKRSDDTASVTGIAEAGKENLGASNSRPSESTVEANSPGDDTVNSASIPVVSGENPLVTPYNGLRHSKDNSDGPAEFAESTKSAESMANPDSNSKGETGKGQDNDMAKATKDSSNSSDGTSSATGDT

SEQ ID NO: 4 - protein, 501 aa, Plasmodium falciparum
ENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 5 - protein, 501 aa, Plasmodium falciparum
ENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 6 - protein, 501 aa, Plasmodium falciparum
ENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 7 - protein, 501 aa, Plasmodium falciparum
ENAIKKTKNQENQLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKIQETYDKVKSKCNDIKNDLIATIKKLEHPYDINNKNDDSYRYDISEEIDDKSEETDDETEEVEDSIQDTDSQHTPSNKKKNDLMQRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 8 - protein, 501 aa, Plasmodium falciparum
ENAIKKTKNQENQLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKIQETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 9 - protein, 501 aa, Plasmodium falciparum; identical to SEQ ID NO: 4

SEQ ID NO: 10 - protein, 10 aa, synthetic construct
  VARIANT 6..10: note = up to 5 H residues may be deleted
HHHHHHHHHH

SEQ ID NO: 11 - protein, 6 aa, synthetic construct
HHHHHH

SEQ ID NO: 12 - protein, 8 aa, synthetic construct
HHHHHHHH

SEQ ID NO: 13 - protein, 4 aa, synthetic construct
EPEA

(19) United States
(12) Patent Application Publication
(10) Pub. No.: US 2024/0408187 A1
(43) Pub. Date: Dec. 12, 2024

(54) METHODS AND COMPOSITIONS FOR TREATING AND PREVENTING MALARIA

(71) Applicants: NOVAVAX AB, Uppsala (SE); NOVAVAX, INC., Gaithersburg, MD (US)

(72) Inventors: Jenny M. REIMER, Uppsala (SE); Louis FRIES, Gaithersburg, MD (US); Gregory GLENN, Gaithersburg, MD (US); Karin LÖVGREN BENGTSSON, Stockholm (SE)

(21) Appl. No.: 18/712,227

(22) PCT Filed: Nov. 22, 2022
(86) PCT No.: PCT/US2022/080334

§ 371 Date: May 21, 2024

Related U.S. Application Data
(60) Provisional application No. 63/281,917, filed on Nov. 22, 2021.

(51) Int. Cl. A61K 39/015; A61K 39/00

(52) U.S. Cl. CPC A61K 39/015; A61K 2039/523; A61K 2039/545; A61K 2039/55577

(57) ABSTRACT

The present disclosure provides immunogenic compositions for inducing immune responses against malaria. The present disclosure also provides novel methods of administering said immunogenic compositions. Disclosed herein are methods and compositions for treating malaria by administering immunogenic compositions.

### CROSS REFERENCE TO RELATED APPLICATIONS

This application claims priority to U.S. Provisional Application No. 63/281,917, filed Nov. 22, 2021. The contents of this application are incorporated by reference in their entirety herein for all purposes.

### REFERENCE TO AN ELECTRONIC SEQUENCE LISTING

The contents of the electronic sequence listing (NOVV_095_01WO_SeqList_ST26.xml; Size: (17,531 bytes; and Date of Creation: Nov. 22, 2022) is herein incorporated by reference in its entirety.

### FIELD OF THE DISCLOSURE

The present disclosure relates to compositions and methods for inducing immune responses against Plasmodium parasites.

### BACKGROUND OF THE INVENTION

Infectious diseases remain a problem throughout the world. Malaria is the leading cause of childhood mortality. As of 2019, there were an estimated 229 million cases of malaria worldwide. In 2019, 409,000 people died from malaria. There is only one approved vaccine for preventing malaria, RTS, S/AS01 (RTS,S) (MOSQUIRIX®). The protection RTS, S provides against malaria wanes overtime.

Plasmodium vivax (P. vivax) causes severe morbidity and mortality globally. P. vivax blood-stage merozoites invade reticulocytes through an essential interaction between the parasites' Duffy-binding protein (PvDBP) and the cell's Duffy antigen/chemokine receptor (DARC). There is a need in the art for new vaccines and methods for preventing malaria.

### SUMMARY OF THE INVENTION

The present disclosure provides immunogenic compositions for inducing immune responses against malaria. The present disclosure also provides novel methods of administering said immunogenic compositions. Disclosed herein are methods and compositions for treating malaria by administering immunogenic compositions. The compositions benefit from use of saponin-based adjuvants that provide particularly enhanced immune responses.

In embodiments, provided herein is an immunogenic composition comprising an antigen of a Plasmodium parasite. In embodiments, provided herein is a method of stimulating an immune response against a Plasmodium parasite in a subject comprising administering the immunogenic composition. In embodiments, the Plasmodium parasite is Plasmodium falciparum, Plasmodium malariae, Plasmodium vivax, Plasmodium ovale, or Plasmodium knowlesi. Typically, P. vivax.

In embodiments, the immunogenic composition comprises an adjuvant. In embodiments, the adjuvant comprises at least two iscom particles, wherein: the first iscom particle comprises fraction A of Quillaja saponaria Molina and not fraction C of Quillaja Saponaria Molina; and the second iscom particle comprises fraction C of Quillaja saponaria Molina and not fraction A of Quillaja saponaria Molina. In embodiments, fraction A of Quillaja saponaria Molina and fraction C of Quillaja saponaria Molina account for about 85% by weight and about 15% by weight, respectively, of the sum of weights of fraction A of Quillaja saponaria Molina and fraction C of Quillaja saponaria Molina in the adjuvant. In embodiments, fraction A of Quillaja saponaria Molina and fraction C of Quillaja Saponaria Molina account for about 92% by weight and about 8% by weight, respectively, of the sum of weights of fraction A of Quillaja saponaria Molina and fraction C of Quillaja Saponaria Molina in the adjuvant. In embodiments, the adjuvant is administered at a dose of about 50 μg.

In embodiments, the method of stimulating an immune response against a Plasmodium parasite comprises administering from about 0.1 μg to about 100 μg of antigen. In embodiments, the method of stimulating an immune response against a Plasmodium parasite comprises administering from about 0.1 μg to about 10 μg of antigen. In embodiments, the method of stimulating an immune response against a Plasmodium parasite comprises administering from about 0.1 μg to about 5 μg of antigen. In embodiments, the method of stimulating an immune response against a Plasmodium parasite comprises administering from about 0.1 μg to about 3 μg of antigen. In embodiments, the method of stimulating an immune response against a Plasmodium parasite comprises administering from about 0.1 μg to about 2 μg of antigen. In embodiments, the method comprises administering a first dose and a second dose of the immunogenic composition. In embodiments, the method comprises administering a third dose of the immunogenic composition. In embodiments, the dose of antigen in the second dose is less than the dose of antigen in the first dose. In embodiments, the dose of antigen in the third dose is less than the dose of antigen in the first dose. In embodiments, the first and second dose comprise the same amount of antigen. In embodiments, the second dose and third dose comprise the same amount of antigen. In embodiments, the first dose and third dose comprise the same amount of antigen. In embodiments, the third dose comprises about 90%, about 80%, about 70%, about 60%, about 50%, about 40%, about 30%, about 20%, or about 10% of the antigen in the first dose. In embodiments, the second dose comprises about 90%, about 80%, about 70%, about 60%, about 50%, about 40%, about 30%, about 20% or about 10% of the antigen in the first dose.

Provided herein is a method of stimulating an immune response against a Plasmodium parasite in a subject comprising administering an immunogenic composition comprising a Duffy Binding Protein (DBP) from a Plasmodium parasite. In embodiments, the DBP is from Plasmodium vivax (“PvDBP”). In embodiments, the DBP comprises region I, region II, region III, region IV, region V, region VI, region VII, or any combinations thereof of PvDBP. In embodiments, the DBP comprises region II of PvDBP (“PvDBPII”). In embodiments, the DBP comprises a polypeptide with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 1-9. In embodiments, the DBP comprises a polypeptide with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 1-3. In embodiments, the DBP comprises a polypeptide with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 4-9. In embodiments, the DBP comprises amino acids 194-521 of SEQ ID NO: 1 or a polypeptide with at least 80%, at least 85%, at least 90%, at least 91%, at least 92%, at least 93%, at least 94% at least 95%, at least 96%, at least 97%, at least 98%, at least 99%, or 100% identity to amino acids 194-521 of SEQ ID NO: 1. In embodiments, the DBP is expressed in Escherichia coli. In embodiments, the immunogenic composition comprises from about 1 μg to about 100 μg of DBP, from about 10 μg to about 100 μg of DBP, from about 25 to about 100 μg of DBP, or from about 35 to about 55 μg of DBP. In embodiments, the immunogenic composition comprises about 2 μg, 5 μg, 10 μg, or 50 μg of DBP. In embodiments, the immunogenic composition comprises an adjuvant. In embodiments, the immunogenic composition comprises from about 1 μg to about 100 μg; from about 25 μg to about 75 μg; or from about 40 μg to about 60 μg of adjuvant. In embodiments, the immunogenic composition comprises about 50 μg of adjuvant. In embodiments, the immunogenic composition comprises about 25 μg of adjuvant. In embodiments, the adjuvant is a saponin adjuvant. In embodiments, the saponin adjuvant comprises at least two iscom particles, wherein: the first iscom particle comprises fraction A of Quillaja saponaria Molina and not fraction C of Quillaja saponaria Molina; and the second iscom particle comprises fraction C of Quillaja saponaria Molina and not fraction A of Quillaja saponaria Molina. In embodiments, fraction A of Quillaja saponaria Molina accounts for 50-96% by weight and fraction C of Quillaja saponaria Molina accounts for the remainder, respectively, of the sum of the weights of fraction A of Quillaja saponaria Molina and fraction C of Quillaja saponaria Molina in the adjuvant. In embodiments, fraction A of Quillaja saponaria Molina accounts for at least 75% by weight and fraction C of Quillaja Saponaria Molina accounts for the remainder, respectively, of the sum of the weights of fraction A of Quillaja saponaria Molina and fraction C of Quillaja saponaria Molina in the adjuvant. In embodiments, fraction A of Quillaja saponaria Molina and fraction C of Quillaja Saponaria Molina account for about 85% by weight and about 15% by weight, respectively, of the sum of weights of fraction A of Quillaja saponaria Molina and fraction C of Quillaja Saponaria Molina in the adjuvant. In embodiments, fraction A of Quillaja saponaria Molina and fraction C of Quillaja saponaria Molina account for about 92% by weight and about 8% by weight, respectively, of the sum of weights of fraction A of Quillaja saponaria Molina and fraction C of Quillaja saponaria Molina in the adjuvant. In embodiments, the Plasmodium parasite is Plasmodium falciparum, Plasmodium malariae, Plasmodium vivax, Plasmodium ovale, or Plasmodium knowlesi. In embodiments, the method comprises administering the immunogenic composition in a prefilled syringe. In embodiments, the method comprises administering a first dose and a second dose of the immunogenic composition. In embodiments, the method comprises administering a third dose of the immunogenic composition. In embodiments, the method comprises administering a fourth dose, fifth dose, sixth dose, seventh dose, eighth dose, ninth dose, or tenth dose of the immunogenic composition. In embodiments, the first dose and second dose comprise about the same amount of DBP. In embodiments, the second dose and third dose comprise about the same amount of DBP. In embodiments, the first and third dose comprise about the same amount of DBP. In embodiments, the first, second, and third doses comprise about the same amount of DBP. In embodiments, the first, second, third, and fourth doses comprise about the same amount of DBP. In embodiments, the amount of DBP in the second dose is less than the amount of DBP in the first dose. In embodiments, the amount of DBP in the third dose is less than the amount of DBP in the first dose. In embodiments, the second, third dose or fourth dose or fifth dose or sixth dose or seventh dose or eighth dose or ninth dose or tenth dose comprises about 90%, about 80%, about 70%, about 60%, about 50%, about 40%, about 30%, about 20%, or about 10% of the DBP in the first dose. In embodiments, the second dose comprises about 90%, about 80%, about 70%, about 60%, about 50%, about 40%, about 30%, about 20%, or about 10% of the DBP in the first dose. In embodiments, the method comprises administering the second dose about 1 month after the first dose. In embodiments, the method comprises administering the second dose about 28 days after the first dose. In embodiments, the method comprises administering the third dose about 56 days after the first dose. In embodiments, the method comprises administering the third dose about 2 months after the first dose. In embodiments, the method comprises administering the third dose about 168 days after the first dose. In embodiments, the method comprises administering the third dose about 6 months after the first dose. In embodiments, the method comprises administering the third dose about 14 months after the first dose. In embodiments, the method comprises administering the third dose about 14 months after the second dose. In embodiments, the method comprises administering the third dose at least 5, 6, 7, 8, 9, 10, 11, 12, 13, 14, 15, 16, 17, 18, 19, 20, 21, 22, 23, or 24 months after the first dose or second dose. In embodiments, the method comprises administering the third dose from about 6 months to about 18 months, from about 9 months to about 18 months, from about 12 months to about 15 months, or from about 12 months to about 18 months after the first or second dose. In embodiments, the method comprises: (i) administering a first dose of the immunogenic composition; (ii) administering a second dose of the immunogenic composition about one month after the first dose; and (iii) administering a third dose of the immunogenic composition about fourteen months after the second dose. In embodiments, the method comprises: (i) administering a first dose of the immunogenic composition; (ii) administering a second dose of the immunogenic composition about one month after the first dose; and (iii) administering a third dose of the immunogenic composition about fourteen months after the first dose. In embodiments, the immunogenic composition comprises about 50 μg of DBP. In embodiments, the immunogenic composition comprises about 50 μg saponin adjuvant. In embodiments, the method prevents malaria with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition. In embodiments, the method prevents malaria with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for at least about 2 months, at least about 2.5 months, at least about 3 months, at least about 3.5 months, at least about 4 months, at least about 4.5 months, at least about 5 months, at least about 5.5 months, at least about 6 months, at least about 6.5 months, at least about 7 months, at least about 7.5 months, at least about 8 months, at least about 8.5 months, at least about 9 months, at least about 9.5 months, at least about 10 months, at least about 10.5 months, at least about 11 months, at least about 11.5 months, at least about 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after administration of the immunogenic composition.

### BRIEF DESCRIPTION OF THE DRAWINGS

FIG. 1 shows immune responses induced by a viral vector formulation using ChAd63 and MVA viral vectors (vvDBP) and an immunogenic composition comprising PvDBPII and a saponin adjuvant (i.e., MATRIX-M™). The composition is denoted “DBP/MM.”) For DBP/MM MD 3 doses of the immunogenic composition were administered each one month apart. For DBP/MM DD, the first two doses of the immunogenic composition were administered a month apart and a third dose was administered 14 months after the second dose (“delayed dosing approach”). The lines on the graph are shown at the median with the interquartile range (IQR). The arrows of the graph denote immunization times. Timepoints from final immunization (FV) are labeled. For the vvDBP P—P—B group, only data from the 2^{nd} vaccination onwards is shown. The DBP/MM delayed dosing regimen showed a greater peak IgG response than the other regimens.

FIG. 2 illustrates enhanced immune responses with the delayed dosing approach of FIG. 1. The graph shows the antibody response associated with immunization. The isotype responses are measured by standardized ELISA 14 days after the final vaccination. The dotted line is shown at the assay cut-off of 5 AU. Lines are shown at the median with min to max. Significant pairwise comparisons from Kruskal-Wallis tests with Dunn's multiple comparisons are shown. The IgG2 and IgA2 response (not shown) were near background. The delayed dosing approach resulted in up to an IgG4 response that was up to 40× higher than the other regimens. The delayed dosing approach also resulted in an IgG1 response that was up to 7× higher than the other regimens.

FIGS. 3A-3B illustrate enhanced immune responses with the delayed dosing approach of FIG. 1. FIG. 3A shows the anti-PvDBPII IgG avidity fourteen days after final vaccination. Avidity was measured using a sodium isothiocyanate (NaSCN) chemical displacement ELISA (Kruskal-Wallis test with Dunn's multiple comparisons). FIG. 3B shows the in vitro growth inhibition activity (GIA %) using a transgenic P. knowlesi strain expressing the Salvador I strain of PvDBP. The DBP/MM DD regimen was able to induce growth inhibitory IgG. The lines are shown at the median with interquartile range (IQR). Delayed dosing improves IgG avidity and functionality.

FIGS. 4A-4E illustrate the immune cells induced by immunization with the delayed dosing approach of FIG. 1. FIG. 4A shows that immunization induces antigen-specific IgA+ CD19+ CD20+ memory B cells. FIG. 4B shows that immunization induces antigen-specific IgG+ CD19+ CD20+ memory B cells. FIG. 4C shows that immunization induces antigen-specific IgM+ CD19+ CD20+ memory B cells. DBP-specific cells are defined as those co-staining with two PvDBPII probes and are shown in Q14. Responses are shown in blue at pre-vaccination and pink post-vaccination. FIGS. 4D-4E show the percentage of memory CD4+ T cells producing Th1 cytokines (IFN-γ, TNF-α, IL-2) (FIG. 4D) or Th2 (IL-5, IL-13)(FIG. 4E) cytokines in response to stimulation with media, PvDBPII (“Med DBP”) peptides or SEB control (right-hand axis) at named timepoints. Lines are shown at the median with IQR. The data indicates that vaccination with PvDBPII induces antigen-specific memory IgA+ and IgG+ B cells and CD4+ T cells.

FIG. 5 shows two different viral vectors encoding a PvDBPII (“PvDBP_RII”) antigen and their administration.

FIG. 6 shows an illustrative delayed dosing regimen of an immunogenic composition comprising PvDBPII and saponin adjuvant (e.g., MATRIX-M™).

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen. The immune response may include formation of antibodies and/or a cell-mediated response. Depending on context, the term “vaccine” may also refer to a suspension or solution of an immunogen that is administered to a subject to produce an immune response. Preferably, vaccines induces an immune response that is effective at preventing infection from a Plasmodium parasite.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

The term “percent identity” in the context of two or more nucleic acid or polypeptide sequences, refers to two or more sequences or subsequences that have a specified percentage of nucleotides or amino acid residues that are the same, when compared. Percentage identity can be calculated using the tools CLUSTALW2, which are available online. The following parameters may be used for CLUSTALW2 Pairwise alignment: Protein Weight Matrix=Gonnet; Gap Open=10; Gap Extension=0.1.

As used herein, the terms “co-formulation mix,” “co-formulation,” “co-formulation vaccine compositions,” “prefilled syringes,” “pre-mix,” refer to vaccine formulations that are prepared for short to long-term storage prior to the time of administration to a subject. Such vaccine formulations contain a combination of antigen and adjuvant in the same container and are prepared in advance of administration. In embodiments, prefilled syringes contain a vaccine formulation comprising a DBP polypeptide (e.g., PvDBPII) and an adjuvant (e.g., a saponin adjuvant, such as a Matrix adjuvant).

### Antigens

In embodiments, the immunogenic compositions disclosed herein comprise part or all of the Duffy Binding Protein (DBP). In embodiments, the DBP is from the Plasmodium parasite. In embodiments, the Plasmodium parasite is Plasmodium falciparum, Plasmodium malariae, Plasmodium vivax, Plasmodium ovale, or Plasmodium knowlesi.

In embodiments, the DBP is from a P. vivax parasite. The DBP from P. vivax is referred to as “PvDBP” herein. In embodiments, PvDBP is a polypeptide having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 1.

PvDBP is divided into seven regions (regions I-VII). In embodiments, the DBP is region I, region II, region III, region IV, region V, region VI, region VII, or combinations thereof, of PvDBP. In embodiments, the DBP comprises region II of PvDBP (referred to herein as “PvDBPII”). PvDBPII is an amino-terminal, cysteine-rich region of PvDBP, which contains the receptor binding domain of PvDBP. PvDBPII binds to the Duffy antigen receptor for chemokines (DARC). Critical binding motifs in PvDBPII have been mapped to a 170 amino acid stretch (amino acids 291-460), which includes cysteines 5-8.

In embodiments, PvDBPII is a polypeptide having at least 80%, at least 85%, at least 90%, at least 91%, at least 92%, at least 93%, at least 94%, at least 95%, at least 96%, at least 97%, at least 98%, at least 99%, or 100% identity to a polypeptide comprising amino acids 194 to 521 of SEQ ID NO: 1.

In embodiments, PvDBPII is a polypeptide having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 2.

In embodiments, PvDBPII is a polypeptide having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 3.

The amino acid sequence of the polypeptides of SEQ ID NOS: 1-3 are found below.

| SEQ ID | |
|---|---|
| NO: | Amino Acid Sequence |
| SEQ ID | MKGKNRSLFVLLVLLLLHKVNNVLLERTIETLLECKNEYVKGENGYKL |
| NO: 1 | AKGHHCVEEDNLERWLQGTNERRSEENIKYKYGVTELKIKYAQMNGK |
| | RSSRILKESIYGAHNFGGNSYMEGKDGGDKTGEEKDGEHKTDSKTDNG |
| | KGANNLVMLDYETSSNGQPAGTLDNVLEFVTGHEGNSRKNSSNGGNPY |
| | DIDHKKTISSAIINHAFLQNTVMKNCNYKRKRRERDWDCNTKKDVCIPD |
| | RRYQLCMKELTNLVNNTDTNFHRDITFRKLYLKRKLIYDAAVEGDLLLK |
| | LNNYRYNKDFCKDIRWSLGDFGDIIMGTDMEGIGYSKVVENNLRSIFGT |
| | DEKAQQRRKQWWNESKAQIWTAMMYSVKKRLKGNFIWICKLNVAVNI |
| | EPQIYRWIREWGRDYVSELPTEVQKLKEKCDGKINYTDKKVCKVPPCQ |
| | NACKSYDQWITRKKNQWDVLSNKFISVKNAEKVQTAGIVTPYDILKQEL |
| | DEFNEVAFENEINKRDGAYIELCVCSVEEAKKNTQEVVTNVDNAAKSQ |
| | ATNSNPISQPVDSSKAEKVPGDSTHGNVNSGQDSSTTGKAVTGDGQNG |
| | NQTPAESDVQRSDIAESVSAKNVDPQKSVSKRSDDTASVTGIAEAGKEN |
| | LGASNSRPSESTVEANSPGDDTVNSASIPVVSGENPLVTPYNGLRHSKDN |
| | SDSDGPAESMANPDSNSKGETGKGQDNDMAKATKDSSNSSDGTSSATG |
| | DTTDAVDREINKGVPEDRDKTVGSKDGGGEDNSANKDAATVVGEDRIR |
| | ENSAGGSTNDRSKNDTEKNGASTPDSKQSEDATALSKTESLESTESGDR |
| | TTNDTTNSLENKNGGKEKDLQKHDFKSNDTPNEEPNSDQTTDAEGHDR |
| | DSIKNDKAERRKHMNKDTFTKNTNSHHLNSNNNLSNGKLDIKEYKYRD |
| | VKATREDIILMSSVRKCNNNISLEYCNSVEDKISSNTCSREKSKNLCCSIS |
| | DFCLNYFDVYSYEYLSCMKKEFEDPSYKCFTKGGFKDKTYFAAAGALLI |
| | LLLLIASRKMIKNDSEEATFNEFEEYCDNIHRIPLMPNNIEHMQPSTPLDY |
| | S |
| SEQ ID | DHKKTISSAIINHAFLQNTVMKNCNYKRKRRERDWDCNTKKDVCIPDR |
| NO: 2 | RYQLCMKELTNLVNNTDTNFHRDITFRKLYLKRKLIYDAAVEGDLLLKL |
| | NNYRYNKDFCKDIRWSLGDFGDIIMGTDMEGIGYSKVVENNLRSIFGTD |
| | EKAQQRRKQWWNESKAQIWTAMMYSVKKRLKGNFIWICKLNVAVNIE |
| | PQIYRWIREWGRDYVSELPTEVQKLKEKCDGKINYTDKKVCKVPPCQN |
| | ACKSYDQWITRKKNQWDVLSNKFISVKNAEKVQTAGIVTPYDILKQELD |
| | EFNEVAFENEINKRDGAY |
| SEQ ID | ERDWDCNTKKDVCIPDRRYQLCMKELTNLVNNTDTNFHRDITFRKLYL |
| NO: 3 | KRKLIYDAAVEGDLLLKLNNYRYNKDFCKDIRWSLGDFGDIIMGTDME |
| | GIGYSKVVENNLRSIFGTGEKAQQHRKQWWNESKAQIWTAMMYSVKK |
| | RLKGNFIWICKINVAVNIEPQIYRWIREWGRDYVSELPTEVQKLKEKCDG |
| | KINYTDKKVCKVPPCQNACKSYDQWITRKKNQWDVLSNKFKSVKNAE |
| | KVQTAGIVTPYDILKQELDEFNEVAFENEINKRDGAYIELCVCSVEEAKK |
| | NTQEVVTNVDNAAKSQATNSNPISQPVDSSKAEKVPGDSTHGNVNSGQ |
| | DSSTTGKAVTGDGQNGNQTPAE |
| | SDVQRSDIAESVSAKNVDPQKSVSKRSDDTASVTGIAEAGKENLGASNS |
| | RPSESTVEANSPGDDTVNSASIPVVSGENPLVTPYNGLRHSKDNSDGPAE |
| | FAESTKSAESMANPDSNSKGETGKGQDNDMAKATKDSSNSSDGTSSAT |
| | GDT |

In embodiments, the DBP is from a P. falpicarum parasite. In embodiments, the DBP from a P. falpicarum parasite is a polypeptide having at least 80%, at least 85% at least 90%, at least 91% at least 92% at least 93% at least 94% at least 95% at least 96% at least 97%, at least 98% at least 99% at least 99.5% or 100% identity to a polypeptide having the amino acid sequence of any one of SEQ ID NOS: 4-9.

| SEQ ID | |
|---|---|
| NO: | Amino Acid Sequence |
| SEQ ID | ENAIKKTKNQENNLTLLPIKSTEEEKDDIKNGKDIKKEIDNDKENIKTNN |
| NO: 4 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKCIAVDAFIKKINETYDKVKSKC |
| | NDIKNDLIATIKKLEHPYDINNKNDDSYRYDISEEIDDKSEETDDETEEVE |
| | DSIQDTDSNHTPSNKKKNDLMNRTFKKMMDEYNTKKKKLIKCIKNHEN |
| | DENKICMDMKNYGTNLFEQLSCYNNNFCNTNGIRYHYDEYIHKLILSVK |
| | SKNLNKDLSDMTNILQQSELLLTNLNKKMGSYIYIDTIKFIHKEMKHIFN |
| | RIEYHTKIINDKTKIIQDKIKLNIWRTFQKDELLKRILDMSNEYSLFITSDH |
| | LRQMLYNTFYSKEKHLNNIFHHLIYVLQMKFNDVPIKMEYFQTYKKNK |
| | PLTQ |
| SEQ ID | ENAIKKTKNQENNLALLPIKSTEEEKDDIKNGKDIKKEIDNDKENIKTNN |
| NO: 5 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKYIAVDAFIKKINEAYDKVKSK |
| | CNDIKNDLIATIKKLEHPYDINNKNDDSYRYDISEEIDDKSEETDDETEEV |
| | EDSIQDTDSNHAPSNKKKNDLMNRAFKKMMDEYNTKKKKLIKCIKNHE |
| | NDFNKICMDMKNYGTNLFEQLSCYNNNFCNTNGIRYHYDEYIHKLILSV |
| | KSKNLNKDLSDMTNILQQSELLLTNLNKKMGSYIYIDTIKFIHKEMKHIF |
| | NRIEYHTKIINDKTKIIQDKIKLNIWRTFQKDELLKRILDMSNEYSLFITSD |
| | HLRQMLYNTFYSKEKHLNNIFHHLIYVLQMKFNDVPIKMEYFQTYKKN |
| | KPLTQ |
| SEQ ID | ENAIKKTKNQENNLALLPIKSTEEEKDDIKNGKDIKKEIDNDKENIKTNN |
| NO: 6 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKCIAVDAFIKKINEAYDKVKSK |
| | CNDIKNDLIATIKKLEHPYDINNKNDDSYRYDISEEIDDKSEETDDETEEV |
| | EDSIQDTDSNHAPSNKKKNDLMNRAFKKMMDEYNTKKKKLIKCIKNHE |
| | NDFNKICMDMKNYGTNLFEQLSCYNNNFCNTNGIRYHYDEYIHKLILSV |
| | KSKNLNKDLSDMTNILQQSELLLTNLNKKMGSYIYIDTIKFIHKEMKHIF |
| | NRIEYHTKIINDKTKIIQDKIKLNIWRTFQKDELLKRILDMSNEYSLFITSD |
| | HLRQMLYNTFYSKEKHLNNIFHHLIYVLQMKFNDVPIKMEYFQTYKKN |
| | KPLTQ |
| SEQ ID | ENAIKKTKNQENQLTLLPIKSTEEEKDDIKNGKDIKKEIDNDKENIKTNN |
| NO: 7 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKCIAVDAFIKKIQETYDKVKSKC |
| | NDIKNDLIATIKKLEHPYDINNKNDDSYRYDISEEIDDKSEETDDETEEVE |
| | DSIQDTDSQHTPSNKKKNDLMQRTFKKMMDEYNTKKKKLIKCIKNHEN |
| | DFNKICMDMKNYGTNLFEQLSCYNNNFCNTNGIRYHYDEYIHKLILSVK |
| | SKNLNKDLSDMTNILQQSELLLTNLNKKMGSYIYIDTIKFIHKEMKHIFN |
| | RIEYHTKIINDKTKIIQDKIKLNIWRTFQKDELLKRILDMSNEYSLFITSDH |
| | LRQMLYNTFYSKEKHLNNIFHHLIYVLQMKENDVPIKMEYFQTYKKNK |
| | PLTQ |
| SEQ ID | ENAIKKTKNQENQLTLLPIKSTEEEKDDIKNGKDIKKEIDNDKENIKTNN |
| NO: 8 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKCIAVDAFIKKIQETYDKVKSKC |
| | NDIKNDLIATIKKLEHPYDINNKNDDSYRYDISEEIDDKSEETDDETEEVE |
| | DSIQDTDSNHTPSNKKKNDLMNRTFKKMMDEYNTKKKKLIKCIKNHEN |
| | DFNKICMDMKNYGTNLFEQLSCYNNNFCNTNGIRYHYDEYIHKLILSVK |
| | SKNLNKDLSDMTNILQQSELLLTNLNKKMGSYIYIDTIKFIHKEMKHIFN |
| | RIEYHTKIINDKTKIIQDKIKLNIWRTFQKDELLKRILDMSNEYSLFITSDH |
| | LRQMLYNTFYSKEKHLNNIFHHLIYVLQMKFNDVPIKMEYFQTYKKNK |
| | PLTQ |
| SEQ ID | ENAIKKTKNQENNLTLLPIKSTEEEKDDIKNGKDIKKEIDNDKENIKTNN |
| NO: 9 | AKDHSTYIKSYLNTNVNDGLKYLFIPSHNSFIKKYSVFNQINDGMLLNEK |
| | NDVKNNEDYKNVDYKNVNFLQYHFKELSNYNIANSIDILQEKEGHLDF |
| | VIIPHYTFLDYYKHLSYNSIYHKSSTYGKCIAVDAFIKKINETYDKVKSKC |
| | NDIKNDLIATIKKLEHPYDINNKNDDSYRYDISEEIDDKSEETDDETEEVE |
| | DSIQDTDSNHTPSNKKKNDLMNRTFKKMMDEYNTKKKKLIKCIKNHEN |
| | DFNKICMDMKNYGTNLFEQLSCYNNNFCNTNGIRYHYDEYIHKLILSVK |
| | SKNLNKDLSDMTNILQQSELLLTNLNKKMGSYIYIDTIKFIHKEMKHIFN |
| | RIEYHTKIINDKTKIIQDKIKLNIWRTFQKDELLKRILDMSNEYSLFITSDH |
| | LRQMLYNTFYSKEKHLNNIFHHLIYVLQMKENDVPIKMEYFQTYKKNK |
| | PLTQ |

Additional suitable antigens are disclosed in the following references which are incorporated by reference herein in their entireties for all purposes: Singh et al. “Malaria vaccine candidate based on Duffy-binding protein elicits strain transcending functional antibodies in a Phase I trial.” NPJ vaccines vol. 3 48. 28 Sep. 2018; Bhardwaj, R. et al. Production of recombinant PvDBPII, receptor binding domain of Plasmodium vivax Duffy binding protein, and evaluation of immunogenicity to identify an adjuvant formulation for vaccine development. Protein Expr. Purif. 136, 52-57 (2015); U.S. Pat. No. 5,993,827; and Chitnis et al. J Exp Med. 1994 Aug. 1; 180(2): 497-506.

In embodiments, the DBP is expressed and purified from an E. coli host cell. In embodiments, the DBP is expressed and purified in an insect cell. In embodiments, the insect cell is a Drosophila A2 cell. In embodiments, the DBP is expressed in a host cell using a fed-batch fermentation strategy. In embodiments, the DBP is purified using chromatography. In embodiments, the chromatography is cation exchange chromatography. In embodiments, the chromatography is anion exchange chromatography. In embodiments, the chromatography is affinity chromatography. In embodiments, the DBP comprises a C-tag and is purified via the C-tag. In embodiments, the DBP is filtered on an anion-exchange support. In embodiments, the anion-exchange support comprises pendant quaternary amine functional groups in a cross-linked polymeric coating. The following references describe expression and purification of DBPs and are incorporated by reference herein in their entireties for all purposes: Singh et al. Npj Vaccines, volume 3, article 48 (2018); Hjerrild et al. Scientific Reports, 6: 30357 (2016); Bhardwaj, R. et al. Production of recombinant PvDBPII, receptor binding domain of Plasmodium vivax Duffy binding protein, and evaluation of immunogenicity to identify an adjuvant formulation for vaccine development. Protein Expr. Purif. 136, 52-57 (2015).

In embodiments, a DBP described herein comprises a N-terminal or C-terminal tag. In embodiments, the N-terminal or C-terminal tag is a polyglutamate tag, a FLAG-tag, a HA-tag, a polyHis-tag (having about 5-10 histidines) (SEQ ID NO: 10), a hexahistidine tag (SEQ ID NO: 11), an 8×-His-tag (having eight histidines) (SEQ ID NO: 12), a Myc-tag, a Glutathione-S-transferase-tag, a Green fluorescent protein-tag, Maltose binding protein-tag, a Thioredoxin-tag, an Fc-tag, or a C-tag. In embodiments, the extension comprises a C-tag. A C-tag comprises the sequence EPEA (SEQ ID NO: 13). In embodiments, the N-terminal or C-terminal tag is removed.

In embodiments, provided herein are nucleic acids encoding any of the DBP described herein. In embodiments, the nucleic acid encoding the antigen of the Plasmodium parasite is extended at the 5′end, 3′ end, or both. In embodiments, the extension encodes a protein used for purification or detection. Non-limiting examples of tags are described throughout this disclosure. In embodiments, the tag is removed.

Advantageously, the immunogenic compositions described herein are able to induce robust humoral immune responses which can inhibit parasite growth in vitro. The present disclosure takes advantage of a delayed boost approach to provide remarkably enhanced immune responses. Delayed dosing of the DBP/MM vaccine results in substantially improved IgG concentrations, avidity and alters the isotype profile, notably increasing IgG4 and IgG1 responses. Thus, in embodiments, a second immunogenic composition is administered after a first composition to boost the response separated by at least 5, 6, 7, 8, 9, 10, 11, 12, 13, 14, 15 or at least 16 months. The first and second compositions may be the same or different.

### Adjuvants

In embodiments, the immunogenic compositions comprise an adjuvant.

In certain embodiments, the compositions disclosed herein may be combined with one or more adjuvants to enhance an immune response. In other embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

In embodiments, the adjuvant is a saponin adjuvant.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In particular embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 10% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In some aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In embodiments, the Fraction A ISCOM matrix accounts for at least 75% by weight and Fraction C ISCOM matrix accounts for the remainder, respectively, of the sums of the weights of Fraction A ISCOM matrix and Fraction C ISCOM in the adjuvant. In embodiments, the Fraction A ISCOM matrix is present at about 92% and Fraction C ISCOM matrix is present at about 8% of the total weight amount of saponin adjuvant in the composition. In a particularly preferred composition, referred to herein as MATRIX-M™, the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In some, compositions other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

In embodiments, the immunogenic composition comprises a Toll-like receptor 4 agonist glucopyranosyl lipid adjuvant-stable emulsion (GLA-SE). This adjuvant is described in the following reference which is incorporated by reference herein in its entirety: Behzad et al. J Infect Dis. 2012 Feb. 1; 205(3): 466-473.

### Excipients

In embodiments, the immunogenic compositions described herein comprise various excipients, buffers, and the like. For example, the immunogenic compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate is present. Histidine may be present about 0.1% (w/v), about 0.5% (w/v), about 0.7% (w/v), about 1% (w/v), about 1.5% (w/v), about 2% (w/v), or about 2.5% (w/v). Sodium chloride, when present, may be about 150 mM. In certain compositions, the sodium chloride may be present in higher concentrations, for example from about 200 mM to about 500 mM. In embodiments, the sodium chloride is present in a high concentration, including but not limited to about 200 mM, about 250 mM, about 300 mM, about 350 mM, about 400 mM, about 450 mM, or about 500 mM.

### Administration and Dosage

Provided herein are novel methods for administering the aforementioned immunogenic compositions. In embodiments, the immunogenic compositions induce an immune response against malaria in a subject in need thereof. In embodiments, the immunogenic compositions induce an immune response against a parasite selected from Plasmodium falciparum, Plasmodium malariae, Plasmodium vivax, Plasmodium ovale, or Plasmodium knowlesi.

In embodiments, the amount of DBP administered per dose ranges from about 0.1 μg to about 100 μg, including all ranges and subranges therebetween. For example, a dose of 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg is administered. In embodiments, each dose of immunogenic composition comprises about 50 μg of DBP.

In embodiments, the immunogenic compositions comprise an adjuvant. In embodiments, the dose of adjuvant ranges from about 1 μg to about 100 μg. For example, in embodiments, the dose of adjuvant is about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg. In embodiments, the adjuvant is a saponin adjuvant. In embodiments, the saponin adjuvant comprises 85% w/w Fraction A ISCOM matrix and 15% w/w Fraction C ISCOM matrix. In embodiments, an immunogenic composition comprises about 50 μg adjuvant.

In embodiments, the immunogenic compositions described herein are administered in a single dose. In embodiments, the immunogenic compositions described herein are administered in multiple doses. For example, in embodiments, one, two, three, four, five, six, seven, eight, nine, or ten doses of the immunogenic composition is administered. These doses are referred to as a first, second, third, fourth, fifth, sixth, seventh, eight, ninth, or tenth dose.

In embodiments, the second, third, fourth, fifth, sixth, seventh, eight, ninth, or tenth dose of an immunogenic composition is administered about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 53 weeks, about 54 weeks, about 55 weeks, about 56 weeks, about 57 weeks, about 58 weeks, about 59 weeks, about 60 weeks, about 61 weeks, about 62 weeks, about 63 weeks, about 64 weeks, about 65 weeks, about 66 weeks, about 67 weeks, about 68 weeks, about 69 weeks, about 70 weeks, about 71 weeks, about 72 weeks, about 73 weeks, about 74 weeks, about 75 weeks, about 76 weeks, about 77 weeks, about 78 weeks, about 79 weeks, about 80 weeks, about 81 weeks, about 82 weeks, about 83 weeks, about 84 weeks, about 85 weeks, about 86 weeks, about 87 weeks, about 88 weeks, about 89 weeks, about 90 weeks, about 91 weeks, about 92 weeks, about 93 weeks, about 94 weeks, about 95 weeks, about 96 weeks, about 97 weeks, about 98 weeks, about 99 weeks, about 100 weeks, about 1 month (e.g., 28 days, 29 days, 30 days, or 31 days), about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administration of the first dose of the immunogenic composition. In embodiments, a second dose of an immunogenic composition is administered about one month after administration of the first dose. In embodiments, the third dose of an immunogenic composition is administered about 1 month or about 2 months after administration of the first dose. In embodiments, the third dose of an immunogenic composition is administered about 14 months or about 15 months after administration of the first or second dose.

In embodiments, the second, third, fourth, fifth, sixth, seventh, eight, ninth, or tenth dose of an immunogenic composition is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, at least about 31 weeks, at least about 32 weeks, at least about 33 weeks, at least about 34 weeks, at least about 35 weeks, at least about 36 weeks, at least about 37 weeks, at least about 38 weeks, at least about 39 weeks, at least about 40 weeks, at least about 41 weeks, at least about 42 weeks, at least about 43 weeks, at least about 44 weeks, at least about 45 weeks, at least about 46 weeks, at least about 47 weeks, at least about 48 weeks, at least about 49 weeks, at least about 50 weeks, at least about 51 weeks, at least about 52 weeks, at least about 53 weeks, at least about 54 weeks, at least about 55 weeks, at least about 56 weeks, at least about 57 weeks, at least about 58 weeks, at least about 59 weeks, at least about 60 weeks, at least about 61 weeks, at least about 62 weeks, at least about 63 weeks, at least about 64 weeks, at least about 65 weeks, at least about 66 weeks, at least about 67 weeks, at least about 68 weeks, at least about 69 weeks, at least about 70 weeks, at least about 71 weeks, at least about 72 weeks, at least about 73 weeks, at least about 74 weeks, at least about 75 weeks, at least about 76 weeks, at least about 77 weeks, at least about 78 weeks, at least about 79 weeks, at least about 80 weeks, at least about 81 weeks, at least about 82 weeks, at least about 83 weeks, at least about 84 weeks, at least about 85 weeks, at least about 86 weeks, at least about 87 weeks, at least about 88 weeks, at least about 89 weeks, at least about 90 weeks, at least about 91 weeks, at least about 92 weeks, at least about 93 weeks, at least about 94 weeks, at least about 95 weeks, at least about 96 weeks, at least about 97 weeks, at least about 98 weeks, at least about 99 weeks, at least about 100 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months after administration of the first or second dose of the immunogenic composition. In embodiments, a second dose of an immunogenic composition is administered at least about one month after administration of the first dose. In embodiments, the third dose of an immunogenic composition is administered at least about 1 month or at least about 2 months after administration of the first dose. In embodiments, the third dose of an immunogenic composition is administered at least about 14 months or at least about 15 months after administration of the first or second dose.

In embodiments, the third, fourth, fifth, sixth, seventh, eight, ninth, or tenth dose of an immunogenic composition is administered from about 6 months to about 18 months, from about 9 months to about 18 months, from about 12 months to about 15 months, from about 12 months to about 18 months, or from about 12 months to about 24 months after the first or second dose. In embodiments, the second dose of an immunogenic composition is administered from about 6 months to about 18 months, from about 9 months to about 18 months, from about 12 months to about 15 months, from about 12 months to about 18 months, or from about 12 months to about 24 months after the first dose.

In embodiments, three doses of an immunogenic composition described herein are administered, a first dose, a second dose, and a third dose. In embodiments, each dose comprises of the immunogenic composition comprises about 50 μg of DBP. In embodiments, each dose of the immunogenic composition comprises about 50 μg of adjuvant. In embodiments, each dose of the immunogenic composition comprises about 50 μg of adjuvant and about 50 μg of antigen. In embodiments, the second dose is administered about 1 month after administration of the first dose and the third dose is administered about 14 months after administration of the second dose. In embodiments, the second dose is administered about 1 month after administration of the first dose and the third dose is administered about 14 months after administration of the first dose.

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

In embodiments, a dose is administered in a volume of about 0.1 mL to about 1.5 mL, for example, about 0.1 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL. In embodiments, the dose is administered in a volume of 0.25 mL. In embodiments, the dose is administered in a volume of 0.5 mL. In embodiments, the dose is administered in a volume of 0.6 mL.

In embodiments, an immunogenic composition may comprise an antigen at a concentration of about 1 μg/mL to about 50 μg/mL, 10 μg/mL to about 100 μg/mL, about 10 μg/mL to about 50 μg/mL, about 175 μg/mL to about 325 μg/mL, about 200 μg/mL to about 300 μg/mL, about 220 μg/mL to about 280 μg/mL, or about 240 μg/mL to about 260 μg/mL.

In embodiments, an immunogenic composition described herein has an efficacy of preventing an infection with malaria. The efficacy may be determined by comparing the number of subjects not administered a malaria immunogenic composition that are diagnosed with malaria (controls diagnosed with malaria) to the number of subjects administered a malaria immunogenic composition that are subsequently diagnosed with malaria. The following equation is used to calculate efficacy: 100×(percentage of controls diagnosed with malaria−percentage of subjects administered the immunogenic composition diagnosed with malaria)/(percentage of controls diagnosed with malaria).

In embodiments, the immunogenic compositions described herein have an efficacy of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100%.

In embodiments, the methods described herein prevent malaria with efficacy from about 50% to about 99% from about 50% to about 95% from about 50% to about 90% from about 50% to about 85% from about 50% to about 80% from about 60% to about 99% from about 65% to about 95% from about 65% to about 90% from about 65% to about 85% from about 69% to about 81% from about 60% to about 95%, from about 60% to about 900%, from about 60% to about 85% from about 60% to about 80% from about 40% to about 99% from about 40% to about 95% from about 40% to about 90% from about 40% to about 85% from about 40% to about 80% from about 40% to about 75% from about 40% to about 70% from about 40% to about 65% from about 40% to about 55% or from about 40% to about 50% for at least about 2 months, at least about 2.5 months, at least about 3 months, at least about 3.5 months, at least about 4 months, at least about 4.5 months, at least about 5 months, at least about 5.5 months, at least about 6 months, at least about 6.5 months, at least about 7 months, at least about 7.5 months, at least about 8 months, at least about 8.5 months, at least about 9 months, at least about 9.5 months, at least about 10 months, at least about 10.5 months, at least about 11 months, at least about 11.5 months, at least about 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after administration of an immunogenic composition described herein.

In embodiments, the methods described herein prevent malaria with an efficacy from about 50% to about 99% from about 50% to about 95% from about 50% to about 90% from about 50% to about 85% from about 50% to about 80% from about 60% to about 99% from about 65% to about 95% from about 65% to about 90% from about 65% to about 85% from about 69% to about 81% from about 60% to about 95% from about 60% to about 90% from about 60% to about 85%, from about 60% to about 80% from about 40% to about 99% from about 40% to about 95% from about 40% to about 90% from about 40% to about 85% from about 40% to about 80% from about 40% to about 75% from about 40% to about 70% from about 40% to about 65% from about 40% to about 55% or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition.

In some embodiments, the disclosure provides co-formulation (i.e., prefilled syringes or pre-mix) strategies for immunogenic compositions comprising a DBP and an adjuvant (e.g., a saponin adjuvant). Typical vaccine administration strategies currently being utilized are bedside mix formulations. That is, vaccine compositions and adjuvants are stored separately and are mixed prior to administration. Pre-mix, co-formulation, or prefilled syringe strategies for vaccine are less common due to the concerns of the stability of the antigens (e.g., a DBP) and their subsequent immunogenic capabilities. The present disclosure provides immunogenic compositions that can be pre-mixed and stored in advance. The disclosed vaccination strategies and formulations may improve the efficiency of vaccination and may reduce the risks of bedside mixing errors, while maintaining the overall safety and immunogenicity.

A variety of containers may be used to store and transport the pre-mix formulations, including syringes for single administrations and plastic ampules. In some instances, plastic ampules can be manufactured using the blow-fill-seal manufacturing technique or method. In general, the blow-fill-seal (BFS) manufacturing method includes extruding a plastic material (e.g., resin) to form a parison, which is then placed into a mold and cut to size. A filling needle or mandrel is then used to inflate the plastic, which in turn, results in a hollow ampule that substantially conforms to the shape of the mold. Once inflated, a desired volume of liquid can be injected into the ampule, the filling needle or mandrel can be removed, and the ampule can be sealed. Accordingly, BFS can be an automated process that can be performed in a sterile environment without direct human intervention.

In some instances, the ability to aseptically manufacture sterile ampules containing a desired liquid can make BFS manufactured ampules particularly well suited for the pharmaceutical industry. BFS technology, however, has not been compatible with all pharmaceutical liquids, products, etc. For example, some known BFS manufacturing methods include delivering the liquid or product into the ampule while the plastic is still relatively hot, which can result in adverse effects to temperature sensitive liquids and/or products such as vaccines, biologics, etc. Advances in cool BFS technology, however, have increased the variety of suitable products, liquids, etc. allowing some vaccines, biologics, and/or other temperature sensitive pharmaceuticals to be contained in BFS ampules.

In some instances, a BFS ampule can have a size, shape, and/or configuration that is at least partially based on a desired use and/or a desired pharmaceutical liquid or dosage that the ampule is configured to contain. For example, some known BFS ampules can include a pierce through top, a twist-off top, a top including a male or female luer, and/or the like. Some known BFS ampules can have a size and/or shape based on volume of the liquid or dosage configured to be disposed therein. In addition, some known BFS ampules can be manufactured in a strip of multiple, temporarily connected ampules, which can increase manufacturing, packaging, and/or storing efficiencies and/or the like.

In embodiments, the immunogenic compositions described herein are provided in pre-filled syringes. When the immunogenic composition is prepared in a pre-filled syringe, an antigen and adjuvant is combined in advance of administration. In embodiments, the pre-filled syringe contains a DBP and an adjuvant (e.g., a saponin adjuvant). In embodiments, the pre-filled syringe contains DBP and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, the pre-filled syringe contains DBP and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the pre-filled syringe contains DBP and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina accounts for at least about 75% by weight and fraction C of Quillaja Saponaria Molina accounts for the remainder of the sum of weights of fraction A of Quillaja Saponaria Molina and fraction C of Quillaja Saponaria Molina in the adjuvant.

### Example S

### Example 1: Delayed Dosing Approaches Enhanced the Immune Response to Protein-Based Vaccine

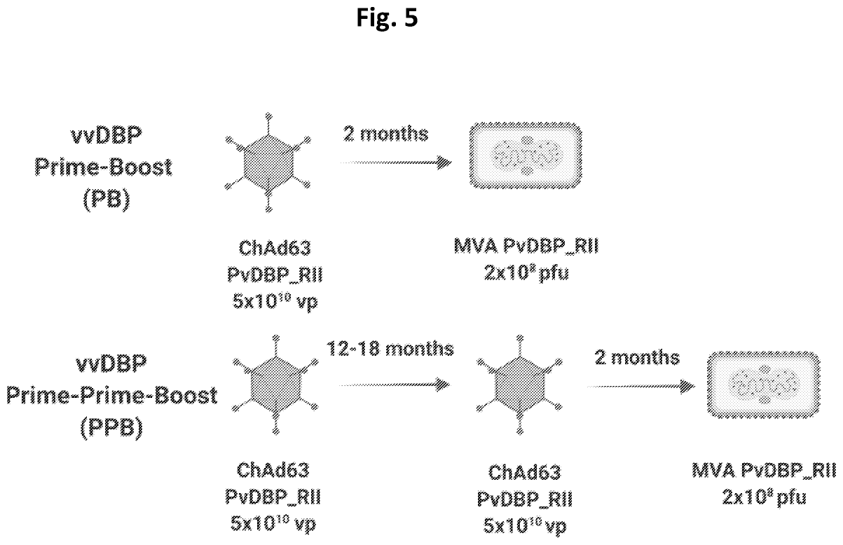

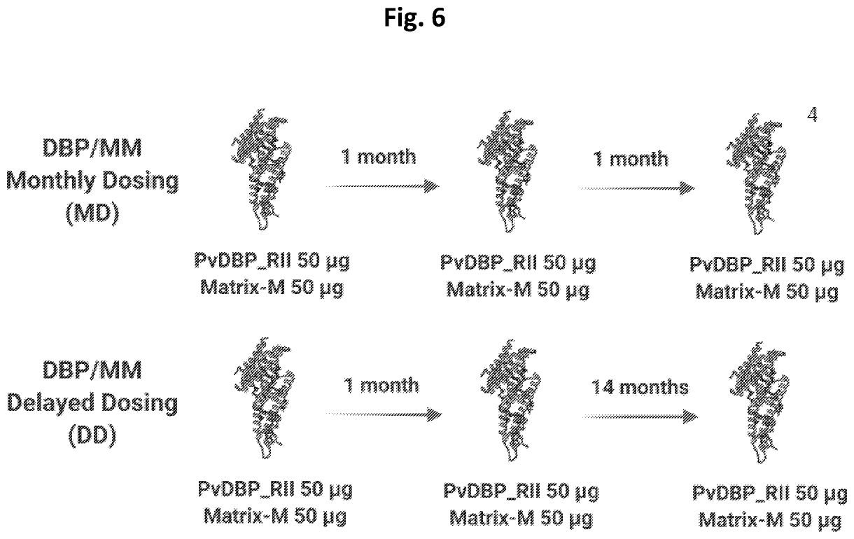

An immunogenic composition comprising PvDBPII and a saponin adjuvant was administering according to the DBP/MM MD (“monthly dosing”) or DBP/MM DD (“delayed dosing”) schedules of FIG. 6. The saponin adjuvant contained 85% w/w Fraction A ISCOM matrix and 15% w/w Fraction C ISCOM matrix. For DBP/MM MD (“Monthly Dosing”) 3 doses were administered each one month apart. For DBP/MM DD (“Delayed Dosing”), the first two doses were a month apart and a third dose was 14 months after the second dose. The immunogenicity of the composition was compared to two viral vector formulations (“vvDBP”): (i) the ChAd63 viral vector encoding PvDBPII and (ii) the MVA viral vector encoding PvDBPII. The vvDBP were administered according to the schedules of FIG. 5.

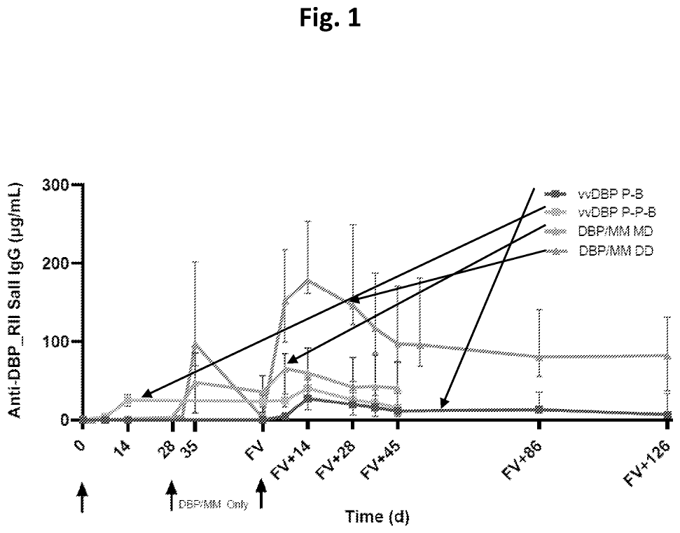

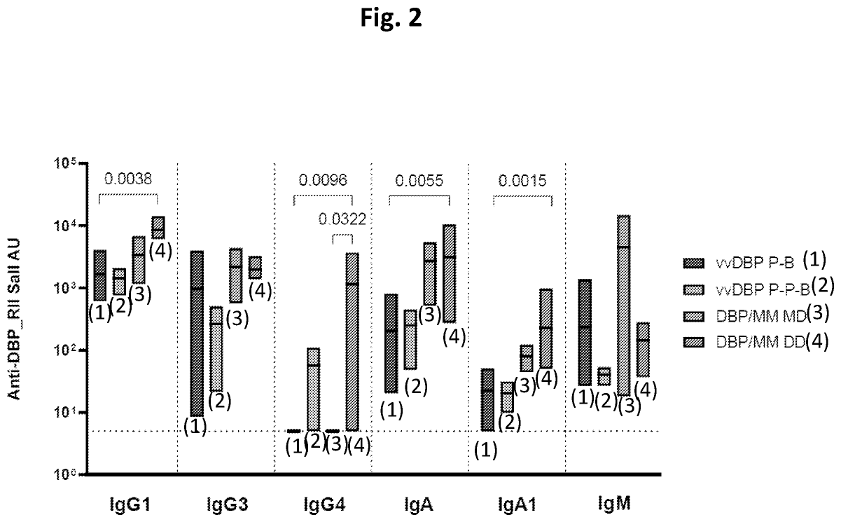

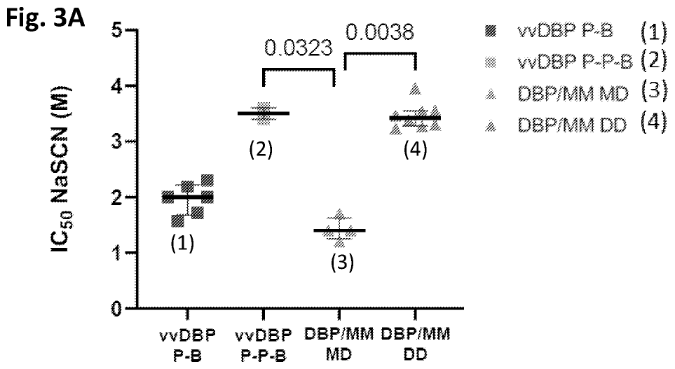

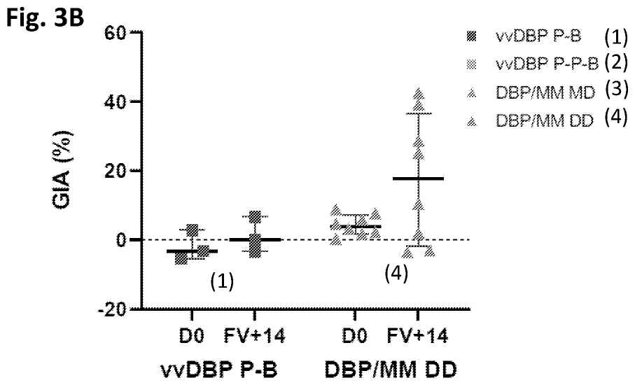

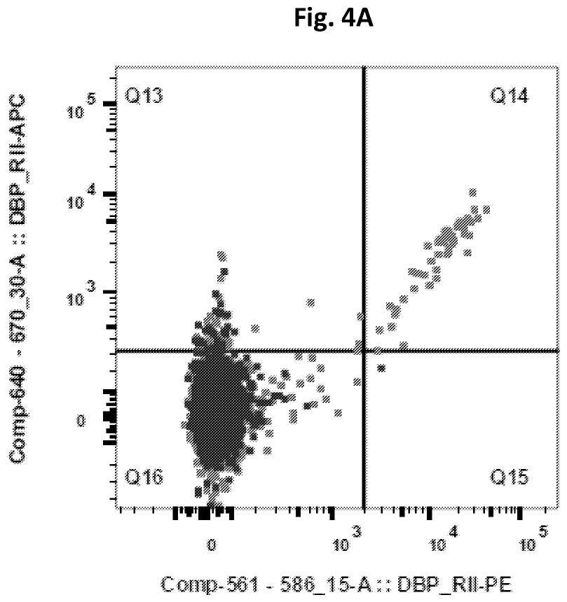

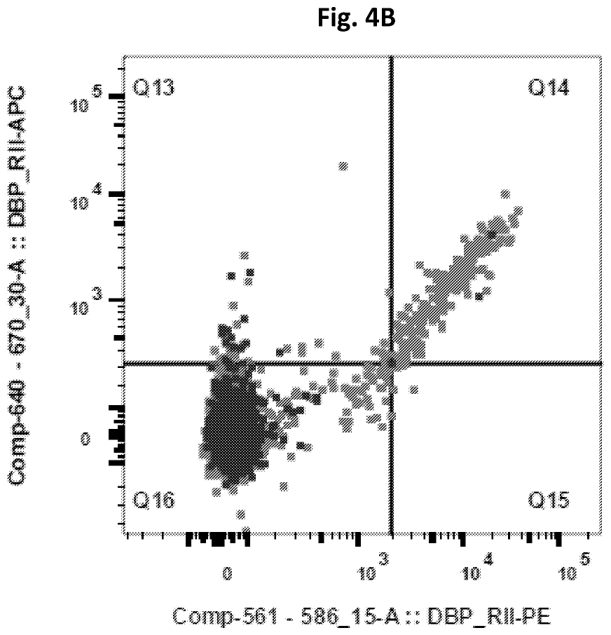

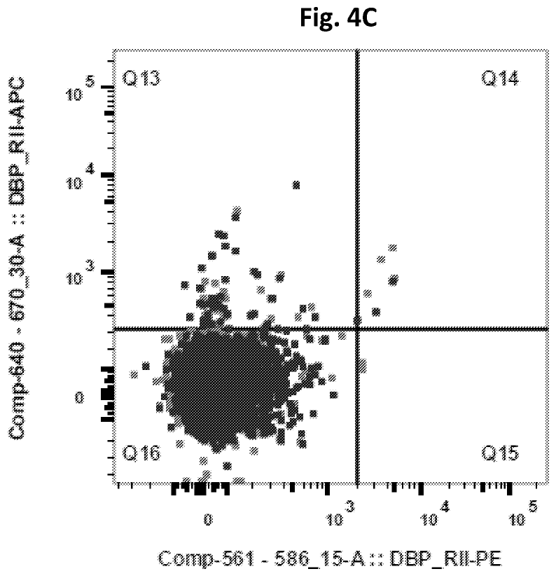

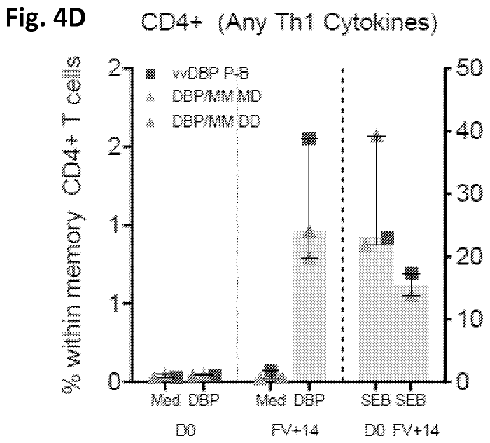

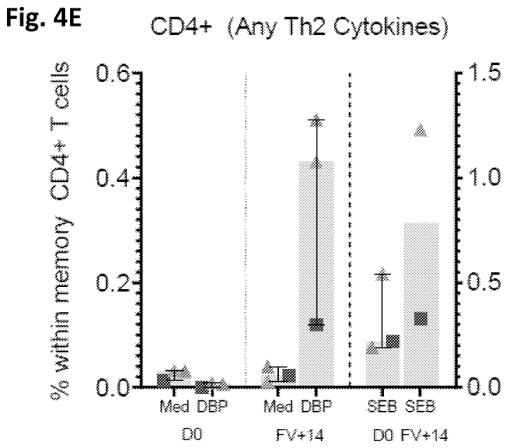

FIG. 1 illustrates shows immune responses induced by each dosing schedule. The immune response obtained by administering the immunogenic compositions containing PvDBPII and a saponin adjuvant was improved compared to the response induced by administering the viral vector formulations. Additionally, the immune response obtained by waiting 14 months to administer the third dose (i.e., “Delayed Dosing”) was remarkably improved compared to Monthly Dosing. Compare FIG. 2 DBP/MM DD vs DBP/MM MD. FIGS. 3A-3B and FIGS. 4A-4E further confirm that delayed dosing enhances the immune response.

### NUMBERED EMBODIMENTS

1. An immunogenic composition comprising a Duffy Binding Protein (DBP) from a Plasmodium parasite.
2. The immunogenic composition of embodiment 1, wherein the DBP is PvDBP.
3. The immunogenic composition of embodiment 1 or 2, wherein the DBP comprises region I, region II, region III, region IV, region V, region VI, region VII, or any combinations thereof of a PvDBP.
4. The immunogenic composition of any one of embodiments 1-3, wherein the DBP comprises region II of PvDBP (“PvDBPII”).
5. The immunogenic composition of any one of embodiments 1-4, wherein the DBP comprises a polypeptide with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 1-9.
6. The immunogenic composition of any one of embodiments 1-4, wherein the DBP comprises amino acids 194-521 of SEQ ID NO: 1 or a polypeptide with at least 80%, at least 85%, at least 90%, at least 91%, at least 92%, at least 93%, at least 94%, at least 95% at least 96%, at least 97%, at least 98%, at least 99%, or 100% identity to amino acids 194-521 of SEQ ID NO: 1.
7. The immunogenic composition of any one of embodiments 1-6, wherein the DBP is expressed in Escherichia coli.
8. The immunogenic composition of any one of embodiments 1-7, comprising an adjuvant.
9. The immunogenic composition of embodiment 8, wherein the adjuvant is a saponin adjuvant.
10. The immunogenic composition of any one of embodiments 8-9, wherein the immunogenic composition comprises from about 1 μg to about 100 μg of adjuvant.
11. The immunogenic composition of any one of embodiments 8-10, wherein the immunogenic composition comprises from about 25 μg to about 75 μg of adjuvant.
12. The immunogenic composition of any one of embodiments 8-11, wherein the immunogenic composition comprises about 50 μg of adjuvant.
13. The immunogenic composition of any one of embodiments 8-12, wherein the immunogenic composition comprises about 25 μg of adjuvant.
14. The immunogenic composition of any one of embodiments 1-13, wherein the immunogenic composition comprises from about 10 μg to about 100 μg of DBP, from about 25 to about 100 μg of DBP, or from about 35 to about 55 μg of DBP.
15. The immunogenic composition of any one of embodiments 1-14, wherein the immunogenic composition comprises about 50 μg of DBP.
16. The immunogenic composition of any one of embodiments 1-15, wherein the Plasmodium parasite is Plasmodium falciparum, Plasmodium malariae, Plasmodium vivax, Plasmodium ovale, or Plasmodium knowlesi.
17. The immunogenic composition of any one of embodiments 8-16, wherein the adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.
   18. The immunogenic composition of embodiment 17, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.
   19. The immunogenic composition of embodiment 17, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.
   20. The immunogenic composition of any one of embodiments 8-19, comprising about 50 μg adjuvant.
   21. The immunogenic composition of any one of claims 1-20, comprising from about 0.1 μg to about 100 μg of DBP; from about 10 μg to about 100 μg of DBP; from about 25 μg to about 75 μg of DBP; or from about 45 μg to about 55 μg of DBP.
   22. The immunogenic composition of any one of claims 1-21, comprising about 50 μg of DBP.
   21. A method of stimulating an immune response against a Plasmodium parasite in a subject comprising administering the composition of any one of embodiments 1-20.
   22. The method of embodiment 21, comprising administering a first dose and a second dose of the immunogenic composition.
   23. The method of embodiment 22, comprising administering a third dose of the immunogenic composition.
   24. The method of any one of embodiments 22-23, wherein the dose of DBP in the second dose is less than the dose of DBP in the first dose.
   25. The method of any one of embodiments 23-24, wherein the dose of DBP in the third dose is less than the dose of DBP in the first dose.
   26. The method of any one of embodiments 23-25, wherein the dose of DBP in the third dose is less than the dose of DBP in the second dose.
   27. The method of any one of embodiments 21-26, wherein the first dose and second dose comprise about the same amount of DBP.
   28. The method of any one of embodiments 23-27, wherein the first dose and third dose comprise about the same amount of DBP.
   29. The method of any one of embodiments 23-27, wherein the second dose and third dose comprise about the same amount of DBP.
   30. The method of any one of embodiments 23-29, wherein the third dose is administered at least 5, 6, 7, 8, 9, 10, 11, 12, 13, 14, 15, 16, 17, 18, 19, 20, 21, 22, 23, or 24 months after the first dose.
   31. The method of any one of embodiments 23-29, wherein the third dose is administered at least 5, 6, 7, 8, 9, 10, 11, 12, 13, 14, 15, or 16 months after the second dose.
   32. The method of any one of embodiments 23-29, wherein the third dose is administered from about 6 months to about 2 years after the first dose.
   33. The method of any one of embodiments 23-29, wherein the third dose is administered from about 6 months to about 2 years after the second dose.
   34. The method of any one of embodiments 23-29, wherein the third dose is administered from about 14 months after the first dose or about 14 months after the second dose.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. This application hereby incorporates by reference the disclosures of U.S. Pat. Nos.

10,729,764, 9,821,046, and 8,821,881 in their entireties for all purposes.

1-38. (canceled)

39. A method of stimulating an immune response against a Plasmodium parasite in a subject comprising administering an immunogenic composition comprising a Duffy Binding Protein (DBP) from a Plasmodium parasite, wherein the DBP comprises a polypeptide with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 1-9.

40. The method of claim 39, wherein the DBP comprises amino acids 194-521 of SEQ ID NO: 1 or a polypeptide with at least 80%, at least 85%, at least 90%, at least 91%, at least 92%, at least 93%, at least 94%, at least 95%, at least 96%, at least 97%, at least 98%, at least 99%, or 100% identity to amino acids 194-521 of SEQ ID NO: 1.

41. The method of claim 39, wherein the DBP is expressed in Escherichia coli.

42. The method of claim 39 wherein the immunogenic composition comprises from about 1 μg to about 100 μg of DBP, from about 10 μg to about 100 μg of DBP, from about 25 to about 100 μg of DBP, or from about 35 to about 55 μg of DBP.

43. The method of claim 39, wherein the immunogenic composition comprises about 2 μg, 5 μg, 10 μg, or 50 μg of DBP.

44. The method of claim 39, wherein the immunogenic composition comprises an adjuvant.

45. The method of claim 44, wherein the immunogenic composition comprises from about 1 μg to about 100 μg; from about 25 μg to about 75 μg; or from about 40 μg to about 60 μg of adjuvant.

46. The method of claim 45, wherein the immunogenic composition comprises about 25 μg of adjuvant or about 50 μg of adjuvant.

47. The method of claim 44, wherein the adjuvant is a saponin adjuvant.

48. The method of claim 47, wherein the saponin adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

49. The method of claim 39, wherein the Plasmodium parasite is Plasmodium falciparum, Plasmodium malariae, Plasmodium vivax, Plasmodium ovale, or Plasmodium knowlesi.

50. The method of claim 39, comprising administering the immunogenic composition in a prefilled syringe.

51. The method of claim 39, comprising administering a first dose and a second dose of the immunogenic composition.

52. The method of claim 51, comprising administering a third dose of the immunogenic composition.

53. The method of claim 51, comprising administering a third dose at least 5, 6, 7, 8, 9, 10, 11, 12, 13, 14, 15, 16, 17, 18, 19, 20, 21, 22, 23, or 24 months after the first dose or second dose.

54. The method of claim 51, wherein the first dose and second dose comprise about the same amount of DBP.

55. The method of claim 51, wherein the amount of DBP in the second dose is less than the amount of DBP in the first dose.

56. The method of claim 51, comprising administering the second dose about 1 month after the first dose.

57. The method of claim 39, comprising:
(i) administering a first dose of the immunogenic composition;
(ii) administering a second dose of the immunogenic composition about one month after the first dose;
(iii) administering a third dose of the immunogenic composition about fourteen months after the second dose.

58. The method of claim 39, wherein the method prevents malaria with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition.
